(19) United States
(12) Plant Patent Application Publication
(10) Pub. No.: US 2004/0139516 P1
(43) Pub. Date: Jul. 15, 2004

(54) LOBELIA PLANT NAMED 'LOBETO'

(76) Inventor: Heinrich Westhoff, Oeding (DE)

Correspondence Address: SYNGENTA BIOTECHNOLOGY, INC., PATENT DEPARTMENT, 3054 CORNWALLIS ROAD, P.O. BOX 12257, RESEARCH TRIANGLE PARK, NC, 27709-2257, US

(21) Appl. No.: 10/342,714

(22) Filed: Jan. 15, 2003

(51) Int. Cl. A01H 5/00

(52) U.S. Cl. PLT/263

(57) ABSTRACT

A new Lobelia plant particularly distinguished by its trailing plant habit, large flower size and an intensive flower color.

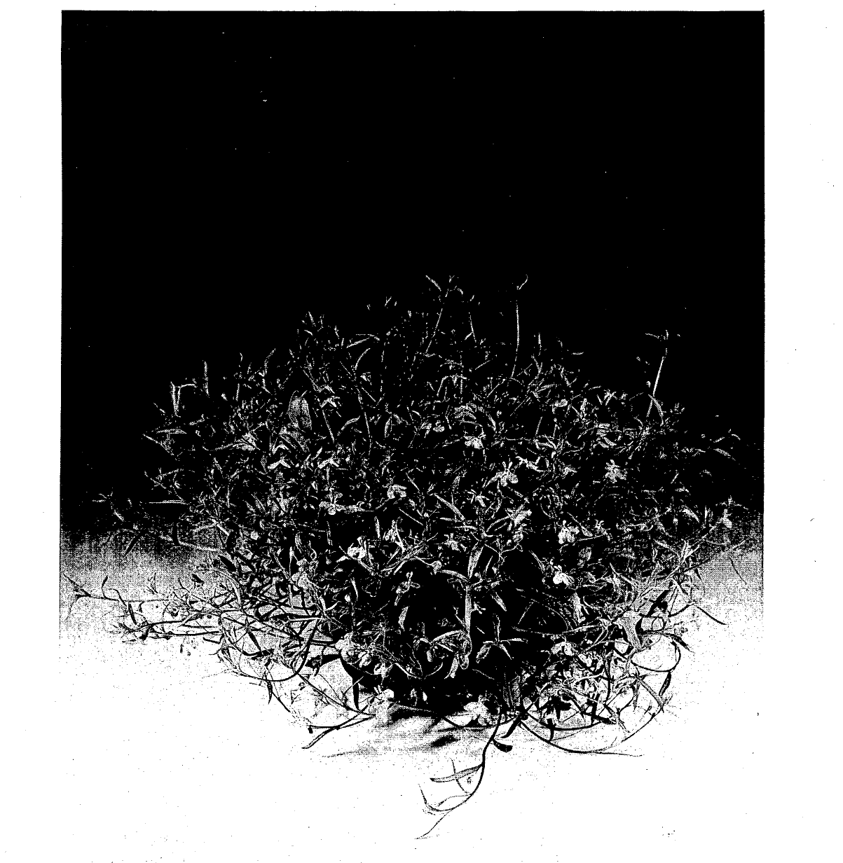

### VARIETAL DENOMINATION

‘Lobeto’

### BACKGROUND OF THE NEW PLANT

The present invention relates to a new and distinct cultivar of Lobelia plant, botantially known as Lobelia erinus. The new cultivar is propagated by cuttings resulting from the cross between female parent, Lobelia Wesstar, ‘PP12.678.P2’ and male parent, wild white Lobelia. The female parent, Lobelia Wesstar has been patented. The male parent, wild white Lobelia has not been patented.

As a result of this cross, the present cultivar was created in 1998 in Sudlohn-Oeding, Germany and has been repeatably asexually reproduced by cuttings in Enkhuizen, Netherlands over a two-year period. It has been found to retain its distinctive characteristics through successive propagations, and this novelty appears to be firmly fixed.

### DESCRIPTION OF THE DRAWING

This new Lobelia plant is illustrated by the accompanying photographic drawing which shows blooms, buds and foliage of the plant in full color, the color shown being as true as can be reasonably obtained by conventional photographic procedures.

### DESCRIPTION OF THE NEW CULTIVAR

The following detailed descriptions set forth the distinctive characteristics of this new Lobelia plant. The data which defines these characteristics were collected from asexual reproductions carried out in Enkhuizen, Netherlands. The plant history was taken on 12 weeks old plants. Color references are primarily to the RHS Colour Chart of The Royal Horticultural Society of London.

Comparison between the new
cultivar ‘Lobeto’ and a similar cultivar

| | ‘Lobeto’ | ‘Blue Star’ |
|---|---|---|
| Plant height | 12 cm | 10 cm |
| Plant width | 35 cm | 35 cm |
| Plant shape | Trailing | Spreading |
| Flower color | | |
| Upper surface | 99B | 95B |
| Lower surface | 97A | 100A |
| Flower tube | 95C | 95C |

The plant:

Classification — botanical.—Lobelia erinus.

Parentage:

Female parent.—Lobelia Wesstar, ‘PP12.678.P2,’ bred in 1996.

Male parent.—Wild white Lobelia.

Plant description:

Growth habit.—Trailing.

Plant height.—12 cm.

Speading area of plant.—35 cm.

Growth rate.—Moderate growth.

Strength.—Good.

Branching character.—Freely branching and lateral branching at every node. Pinching will enhance branching. Branches strong and flexible.

Blooming period.—From May until October.

The stem:

Diameter.—1.5 mm.

Texture.—Smooth, slightly pubescent, glossy.

Color.—RHS 147A.

Length of internode.—15 mm, depending on the light conditions where the plant is propagated.

Shape.—(Tri)angular.

The foliage:

Shape.—Leaves simple, generally symmetrical and flat. Broadly ovate with rounded apex, obtuse to attenuate base and crenate margin.

Texture.—Slightly pubescent, glossy.

Length.—Young 36 mm, fully expanded 54 mm.

Width.—Young 10 mm, fully expanded 24 mm.

Color — upper side.—RHS 147A.

— lower side.—RHS 79B.

The flower:

Type and habit.—Flowers form at apical axils, usually one flower per axil. Flowers labiate, upper lip consisting of two small petals and lower lip consisting of three larger and broader petals. Petals are fused at base into a tube. Freely and continuously flowering. Flowers face outward. Flowers persistent. Texture, slightly pubescent. Oblong shape with acute apex and entire margin.

Flower — width.—upper petals 2 mm, lower petals 6 mm.

— length.—Upper petals 5 mm, lower petals 11 mm.

— Depth.—7 mm.

Texture.—Smooth, pubescent.

Buds — length.—10 mm.

— diameter.—3 mm.

— form.—Tubular.

— Color.—Light green.

Flower color — upper surface.—RHS 99B.

— lower surface.—RHS 97A.

— Flower tube.—RHS 95C.

Sepals — No. of sepals.—5.

— length.—6 mm.

— width.—0.5 mm.

— texture.—Smooth, slightly pubescent.

— color.—RHS 147B.

Flower stem — length.—25 mm.

— angle.—Upright.

— strength.—Rigid, flexible.

— color.—RHS 147A.

Flower throat — color.—Upper RHS 97A Lower RHS 97A, spots RHS 99A.

— texture.—Whiskered.

Reproductive organs:

Androecium — stamen number.—5.

— anther shape.—Oblong.

— anther length.—2 mm.

— Pollen color.—RHS 202B.

— Amount of pollen.—Moderate.

Gynoecium — pistil length.—10 mm.

— stigma shape.—Bilobate.

— stigma diameter.—1 mm.

— stigma color.—N87A.

— style color.—Light green.

— ovary color.—RHS 147A.

Seed development: Some seed development has been observed on plants of the new Lobelia.

Disease resistance: Under commercial conditions, plants of the new Lobelia are resistant to fungal pathogens common to Lobelia.

What is claimed is:

1. A new and distinct variety of Lobelia plant, substantially as illustrated and described herein.
